(19) United States
(12) Patent Application Publication
(10) Pub. No.: US 2024/0033436 A1
(43) Pub. Date: Feb. 1, 2024

(54) Needle-Free Injection System

(71) Applicant: NovaXS Biotech Corp., Irvine, CA (US)

(72) Inventors: Tianyi Xing, Irvine, CA (US); Rui Su, Irvine, CA (US); Tyler Panian, Irvine, CA (US); James Young, Irvine, CA (US)

(21) Appl. No.: 18/359,971

(22) Filed: Jul. 27, 2023

Related U.S. Application Data
(60) Provisional application No. 63/369,941, filed on Jul. 30, 2022.
(60) Provisional application No. 63/369,943, filed on Jul. 30, 2022.
(60) Provisional application No. 63/369,944, filed on Jul. 30, 2022.

(51) Int. Cl. A61M 5/30; A61M 5/31

(52) U.S. Cl. CPC A61M 5/30; A61M 5/31; A61M 2005/3126

(57) ABSTRACT

An injection system includes an injector device and a base station. The injector device includes a cartridge. The injector device further includes an injector body coupled to the cartridge at a first end of the injector body, the injector body comprising a plug at a second end of the injector body. The base station is configured to couple to the plug, wherein the base station comprises a motor, and wherein rotation of the motor draws medicine from a vial into the cartridge, and wherein a volume of the medicine drawn from the vial into the cartridge is in excess of an set dosage.

### CROSS-REFERENCE TO RELATED APPLICATIONS

This application claims priority to (i) U.S. Provisional Application No. 63/369,941 entitled “Needle-Free Injection System,” filed on Jul. 30, 2022, (ii) U.S. Provisional Application No. 63/369,943 entitled “Needle-Free Injection System,” filed on Jul. 30, 2022, and (iii) U.S. Provisional Application No. 63/369,944 entitled “Needle-Free Injection System,” filed on Jul. 30, 2022, the contents of each of which are hereby incorporated by reference in their entirety.

### FIELD OF THE DISCLOSURE

Aspects of the present disclosure relate to an injection system for a needle-free, high pressure injection. More particularly, aspects of the present disclosure include an injector device compatible with a base station, the base station being configured to load medicine from a vial into the injector device and further prepare the injector device for injection. Additional aspects of the present disclosure include an injector device compatible with a vertical alignment feature to ensure proper injection by the user.

### BACKGROUND

Many medical conditions require detailed and, often complicated, injection regimens. These regimens may necessitate frequent trips to the doctor and/or at-home treatments. Frequent trips to the doctor can be time-consuming and inconvenient. And at-home treatments can be painful, expensive, and prone to error and non-compliance from the patient or person administering the treatment. Additionally, traditional injection techniques require needles, which can cause discomfort and even fear in patients.

### SUMMARY

In a first aspect of the present disclosure, an injection system is described. An injection system includes an injector device and a base station. The injector device includes a cartridge and an injector body coupled to the cartridge at a first end of the injector body. The injector body includes a plug at the second end of the injector body. The injector device also includes a spring coupled to the cartridge. The base station is configured to electrically couple to the plug. The base station includes a motor. Rotation of the motor transmits power to the injector device compressing the spring and drawing medicine from a vial into the cartridge.

In a second aspect of the present disclosure, a needle-free injection device is described. The needle-free injection device includes an injector body. The injection device also includes a cartridge coupled to the injector body and having a first height and comprising an orifice. The injector device also includes a cap coupled to the injector body having a second height and including an outer rim, wherein the outer rim surrounds the cartridge, and wherein the first height is greater than the second height.

In a third aspect of the present disclosure, a needle-free injection system is described. The needle-free injection system includes a needle-free injector device and a base station. The injector device includes a cartridge. The injector device further includes an injector body coupled to the cartridge at a first end of the injector body, the injector body comprising a plug at a second end of the injector body. The base station is configured to couple to the plug, wherein the base station comprises a motor, and wherein rotation of the motor draws medicine from a vial into the cartridge, and wherein a volume of the medicine drawn from the vial into the cartridge is in excess of an set dosage.

These as well as other aspects, advantages, and alternatives, will become apparent to those of ordinary skill in the art by reading the following detailed description, with reference where appropriate to the accompanying drawings.

### BRIEF DESCRIPTION OF THE FIGURES

The above, as well as additional, features will be better understood through the following illustrative and non-limiting detailed description of example embodiments, with reference to the appended drawings.

FIG. 1A illustrates a front view of an injector device of an injection system, according to an example embodiment.

FIG. 1B illustrates a cross-sectional view of the injector device of FIG. 1A, according to an example embodiment.

FIG. 2 illustrates a perspective view of a base station of an injection system, according to an example embodiment.

FIG. 3 illustrates a side view of the base station of FIG. 2 with a portion of the base station cut away, according to an example embodiment.

FIG. 4A illustrates a perspective view of an injector device of an injection system, according to an example embodiment.

FIG. 4B illustrates a top, side, and perspective view of a cap of the injector device of FIG. 4A, according to an example embodiment.

All the figures are schematic, not necessarily to scale, and generally only show parts that are necessary to elucidate example embodiments, wherein other parts may be omitted or merely suggested.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the Figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Reference to, e.g., a “first” item does not require or preclude the existence of, e.g., a “second” or higher-numbered item. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, device, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

As used herein, with respect to measurements, “about” means +/−5%.

As used herein, with respect to measurements, “substantially” means +/−5%.

Embodiments of the present disclosure relate to a high-pressure, needle-free, injector device. More particularly, this injector device applies high-pressure to a medicine cartridge attached onto the injector device and ejects the medication through a small orifice. The thin jet of medicine ejected from the orifice penetrates the skin and delivers the drug to the intended area. Sensors integrated into the injection system tracks injection data, such as injection time and injection dosage. The injection system can also connect to a user's mobile device and/or server, by way of a wired and/or wireless connection. Thus, the user can create a profile where this injection data can be stored and accessed.

The injection system includes a base station configured to load or prime the injector device. Namely, a motor within the base station allows for medicine to be loaded into from a vial into the cartridge for injection. This base station loads medicine into the cartridge attached onto the injector in a few seconds. This is desirable, as it reduces the time and effort required by the user during the loading process. The base station design is both durable and portable. Additionally, the light-weight design of the base station reduces the weight of the entire package to more easily accommodate transporting the injection system. Further, the base station is relatively inexpensive to replace, reducing the cost for a user.

Additionally, aspects of the present disclosure, such as the vertical alignment mechanism allow the user to properly inject themselves without others' help. Namely, the cartridge is surrounded by a cap designed for assisting a user to align the cartridge properly against the user's skin (e.g., substantially perpendicular to a plane of the user's skin).

Moreover, aspects of the present disclosure provide automatic medicine drawing and air expelling. Namely, before injection, the user can connect a vial to a vial adaptor and thus to the orifice of the cartridge, which is also used to expel the medicine from the cartridge. The user can set the intended dosage by way of a user interface on the base station. Medicine can be drawn from the vial into the cartridge by way of a motor in the base station. Examples of the present disclosure may draw more medicine from the vial into the cartridge than the set dosage. A vibrating motor within the base station can then be utilized to expel excess air and medicine from the cartridge.

A website or mobile phone application for a user device, such as a smartphone, serves as a portal for the user to the world of digitally connected healthcare. When the treatment cycle starts, a comprehensive step-by-step video tutorial will be provided to the user to properly educate the user before using the device. The tutorial can be easily customized for specific treatment if needed, so the knowledge of the combination of the device and specific medicine will be correctly transferred. Furthermore, the mobile application can provide reminders to the user of the injection schedule, provide visual feedback for the user's adherence pattern, and remind the user to order the next batch of drugs in advance. The application also helps the user to record and report side effects, and then connect the user to a physician if needed.

Further, the website and/or mobile application can provide physicians access to the user's injection adherence data and reported side effects. The adherence to at-home injection is no longer inaccessible to healthcare providers. Thus, physicians can make informed decisions when needed with readily available injection data and injection history of the user/patient.

### I. Injection System

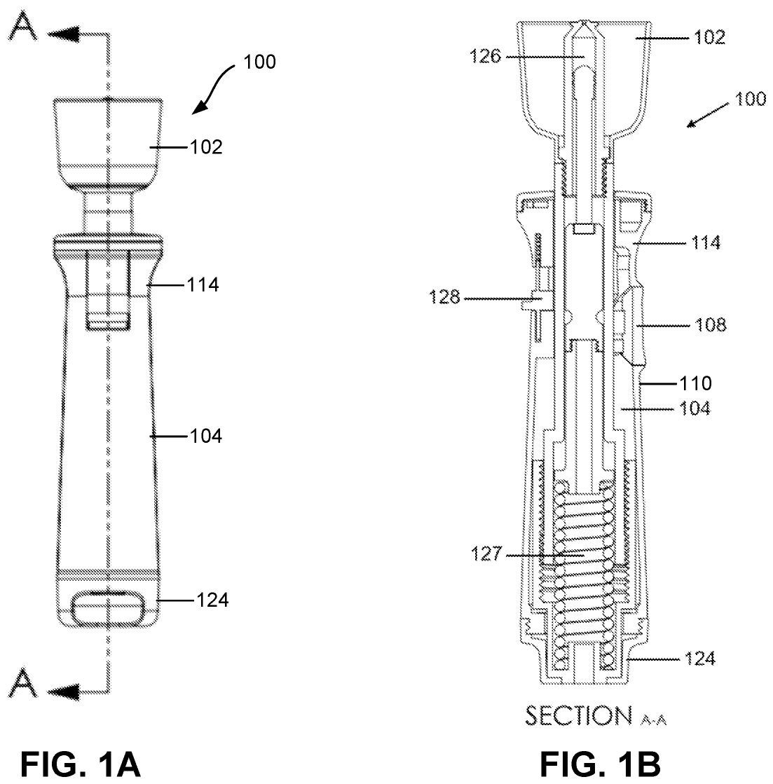

Now referring to FIGS. 1A and 1B, an injector device 100 includes a cap 102, a cartridge 126, and an injector body 104. The injector body 104 connects to a base station 106. The injector body 104 includes a trigger button 108 and an injection indicator 110. The injector body 104 also includes a spring 127 suitable for applying pressure to the medicine in the cartridge 126.

The cartridge 126 includes a small orifice on the top to draw and expel medicine. The cartridge 126 is configured to connect to a vial adaptor, allowing for the withdrawal of medicine from an external vial into the cartridge.

In examples, the injector device 100 can house the vial of medicine. This vial can be replaced and reloaded by the user. In some examples, the injector device 100 can include a sensor to detect when the vial level is low, prompting the user to order more and/or visit a physician.

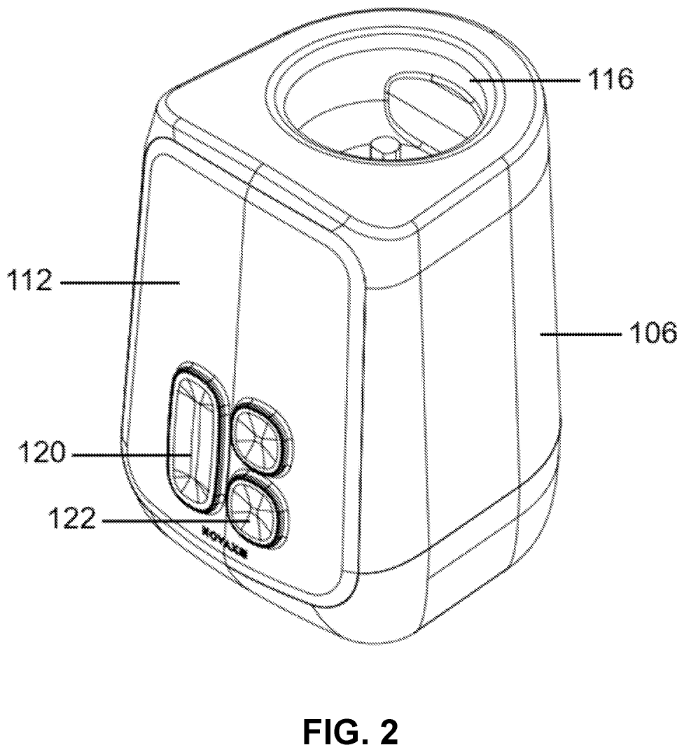

In practice, the user can plug the injector device 100 into the base station 106 to charge, load, and prime the injector device 100. Once the user has indicated the appropriate dosage (as described in more detail with respect to FIG. 2), medicine is drawn from the vial into the cartridge 126. In examples, the cartridge 126 can have a volume of 1 mL, however many examples of possible cartridge sizes are possible.

The injector device 100 also includes a spring suitable for applying pressure to the medicine in the cartridge 126. In the preparation stages, the spring can be compressed by way of a motor in the base station 106.

In other examples, the user can manually load the medicine into the cartridge 126 themselves using a vial adapter. The user can overdraw the medicine, with respected to the set dosage, and can expel air manually. To expel air manually, the user can twist the cartridge 126 onto the injector body 104 and the excess medicine and air will be expelled manually by the preset detent.

In practice, when the injector device 100 is primed by the base station 106 (as described in more detail with respect to FIG. 2) and ready for injection, the trigger button 108, which is initially flush with the injector body 104, can protrude out from the injector body and provide an indication the injector device 100 is ready for injection. The injector body 104 also contains a safety switch 128 opposite the trigger button 108. The safety switch 128 must be activated by sliding the switch towards the tip of the injector in the direction of the cartridge 126 before the trigger button 108 can trigger an injection.

In some examples, the trigger button 108 can include a light indication (for example, a light emitting diode (LED) on or surrounding the trigger button 108) that turns on to indicate the injector device 100 is ready for injection. In these examples, the LED light can include a color (e.g., green) to indicate the ready status of the injector device 100. In other examples, the trigger button 108 can include embossed text on or near the trigger button 108 to indicate the ready status of the injector device 100. For instance, the injector body 104 can include embossed text, such as “READY”, next to or below the trigger button 108 that lights up when the injector device 100 and trigger button 108 are ready.

In examples, the injector body 104 includes an injection indicator 110 that provides the loading state of the injector device 100. The injection indicator 110 can be a mechanical screen that provides different states of the injector device 100. For instance, in some examples, the injection indicator 110 can show a gray line or bar to indicate that the injector device 100 is not loaded and has not fired (i.e., ejected the medicine from the vial). The injection indicator 110 can show a red bar on the injection indicator 110 to indicate that the injector device 100 is loaded, but has not fired. And the injection indicator 110 can show a green check-mark to indicate that the injector device 100 has successfully fired. In examples, the injection indicator 110 can reset to the gray bar when plugged back into the base station 106.

Once the injector device 100 is primed and ready, the user may initiate the injection by placing the cartridge 126 against the user's skin, sliding the safety switch 128, and pressing the trigger button 108. Pressing the trigger button 108 causes the spring 127 to release. The spring 127 applies a high pressure to the medicine in the cartridge 126 causing a thin jet of medicine to expel out of the orifice of the cartridge 126.

The pressure of the thin jet of medicine penetrates the user's skin delivering the medicine to the intended area, thus allowing for subcutaneous injection. Further, in examples, the pressure of the thin jet of medicine allows for intramuscular injections. In example implementations, the medicine can be injected up to 40 mm into a user's body, however, many injection depths are possible.

In examples, the injector body 104 can include a gradual recess, or shoulder 114, at or near where the injector body 104 connects to the cap 102. In practice, the contour of the shoulder 114 helps to create a secure and ergonomic grip for the user. This can assist with stabilization of the injector device 100 before and during injection. Additionally, in examples, the contour of the shoulder 114 is such that it can receive the cap 102 to cover and protect the trigger button 108.

In some examples, the injector device 100 includes an external computing device which includes a communication link. The communication link can take the form of any wired connection (e.g., Ethernet, or USB cable) or wireless connection (e.g., Bluetooth®) over which the computing device and a user device (e.g., computer, smartphone, etc.) can engage in communication. For example, the computing device can transmit injection data to a database storing the user's injection information and history. Further, the communication link of the injector device 100 can transmit data to and receive data from a server (e.g., a cloud server), so that user information can be stored and readily accessed. Additionally or alternatively, the communication link of the base station 106 can transmit and receive data from the injector device 100.

In examples, the injector device 100 includes a rechargeable battery. This rechargeable battery can last up to 30 days on a three-injection-per-day regiment. The base station 106 is configured to charge the rechargeable battery.

Now referring to FIG. 2, the injector device 100 unplugged from the base station 106. In examples, the injector device 100 includes a plug 124. The base station 106 includes a receiving port 116 or socket. The receiving port 116 is configured to receive the plug 124 in order to stabilize the injector device 100 during recharging, loading, and priming of the device. More particularly, the plug 124 and receiving port 116 include mating mechanical connectors. These mating mechanical connectors help facilitate a secure mechanical connection for stability during charging and loading medicine from the vial to the cartridge.

The receiving port 116 is configured to receive the plug 124 in order to stabilize the injector device 100 during recharging, loading, and priming of the device. More particularly, the plug 124 and receiving port 116 include mating mechanical connectors. These mating mechanical connectors help facilitate a secure mechanical connection for stability during charging and loading medicine from the vial to the cartridge.

In some examples, the receiving port 116 can be configured to further electrically coupled to the plug 124 to facilitate the recharging of the injector body 104. Namely, the base station 106 can include an electrical connector. The electrical connector is configured to recharge the battery of the injector body 104, when coupled, by way of the mating connectors of the receiving port 116 and the plug 124.

The motor of the base station 106 is further configured to compress the spring 127 of the injector device 100 and to draw medicine from the vial (not shown) into the cartridge 126 by way of the orifice. More particularly, the spring 127 within the injector device 100 can be compressed to prepare for injection. In some examples, the motor can turn a first direction to prime the injector device 100 (e.g., compress the spring 127) and second direction to draw medicine into the cartridge 126 from the vial.

Further the base station 106 includes a digital screen 112. In examples, the digital screen 112 includes a dosage indicator. The digital screen 112 can additionally include dosage adjustment buttons 120. The dosage adjustment buttons 120 allow the user to easily adjust the dosage of the medicine, for example, in 0.1 mL increments.

Once the user has set the appropriate dosage via the dosage adjustment buttons 120, the user can press the initiate button 122 to begin drawing the medicine from the vial to the cartridge 126.

In some example implementations, the injector device 100, by way of the base station 106, can load more medicine than the indicated dose into the cartridge 126. The injector device 100 can then expel excess medicine and air from the cartridge 126 before injection.

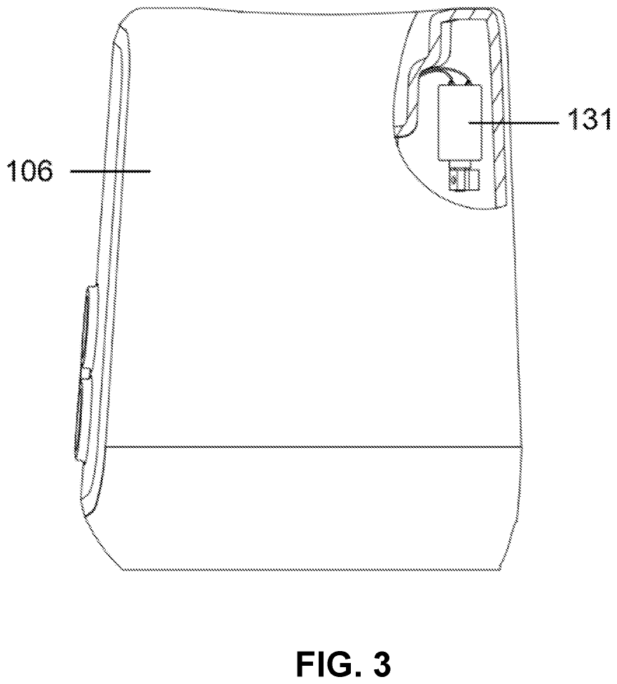

In example embodiments, a slight excess of medicine is drawn from the vial into the cartridge 126 with respect to the indicated dosage. Once the medicine is loaded into the cartridge 126, the injector device 100 can expel excess air and medicine from the cartridge 126 in preparation for injection by way of a vibrating motor 131, as shown in FIG. 3. The vibrating motor 131 applies sufficient mechanical force to the connected injector body 104 to vibrate any contained bubbles within the cartridge 126 in order to bring air contained within the drawn medicine to the tip of the cartridge 126. The excess medicine and air can be expelled by the injector using the motor contained within the base station 106.

In examples, an excess associated with a percentage of the set dosage is drawn from the vial (e.g., 5%-10% extra medicine is drawn than the set dosage). In other examples, a predetermined set volume is drawn in excess of the set dosage (e.g., 0.05 mL extra medicine is drawn than the set dosage).

In some examples, the base station 106 includes a computing device which includes a communication link. The communication link can take the form of any wired connection (e.g., Ethernet, or USB cable) or wireless connection (e.g., Bluetooth®) over which the computing device and a user device (e.g., computer, smartphone, etc.) can engage in communication. For example, the computing device can transmit information to a database storing the user's injection information and history. Further, the communication link of the base station 106 can transmit data to and receive data from a server (e.g., a cloud server), so that user information can be stored and readily accessed. Additionally or alternatively, the communication link of the base station 106 can transmit and receive data from the injector device 100.

In examples, the cartridge 126 can be removed and the cap 102 can twist and/or slide down along the length of the injector body 104. The outer portion of the cap 102 covers and protects the trigger button 108. This helps prevents a user from inadvertently pressing the trigger button 108, thus potentially wasting medicine and injuring the user.

Additionally, the cartridge 126 includes an orifice where medicine is expelled from the injector device 100 once the injector device 100 is primed and loaded with the indicated dosage. In some examples, the cartridge 126 can be removed and replaced by the user. Further, in examples, the cartridge 126 has an orifice positioned within and substantially concentric to the outer portion of the cap 102 when in the injection position.

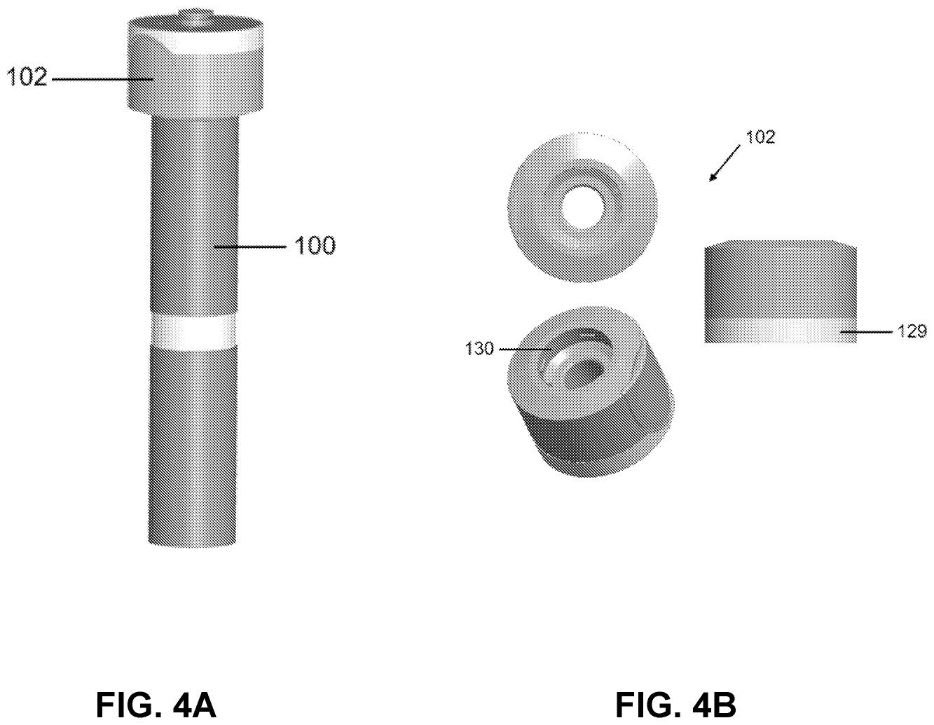

Further, as shown in FIG. 4A, the cartridge 126 extends further away from the injector device 100 than outer portion of the cap 102 in an injection position, such that there is a recess between the cartridge 126 and the top of the outer portion of the cap 102. This helps with alignment for injection, as the outer portion of the cap 102 helps to ensure the injector device 100 is perpendicular to the surface of the skin. In some examples, the configuration of the cap 102 helps ensure that the injector device 100 is vertically aligned, or substantially vertically aligned.

Further, when in use, the recess between the cartridge 126 and the outer rim of the cap 102 helps the user press the cartridge 126 firmly against the user's skin which helps ensure a proper injection. Namely, the user is instructed to ensure that the outer rim of the cap 102 makes contact with the user's skin. In doing so, the cartridge 126 will be pressed more firmly against the user.

The larger radius of the cap 102 helps ensure the injector device 100 is balanced and stable. This also ensures that cartridge 126 is firmly pressed against the user's skin, which also helps prevent accidental firing of the injector device 100.

Dimensions of the cap 102 may vary in different examples devices. For instance, in some examples, the circumference of the outside of the cap 102 may be greater than other embodiments. Similarly, in some examples, the difference heights of the outer rim of the cap 102 and the cartridge 126 (i.e., the height of the recess) may be greater. In these examples, a greater height of the recess will cause the user to press the cartridge 126 more firmly into the user's skin.

A vertical alignment feature of the cap 102 and the safety switch 128 of are used within the needle-free injector to improve the safety and effectiveness of the injection process. The vertical alignment feature of the cap 102 allows the user to properly inject themselves without others' help. Namely, the cartridge 126 is surrounded by a cap 102 designed for assisting a user to align the cartridge 126 properly against the user's skin (e.g., substantially perpendicular to a plane of the user's skin).

Further, in some example embodiments as shown in FIG. 4B, the cap 102 and/or cartridge 126 may include sensors (e.g., pressure and/or touch sensors) around the skin contacting surface 129 to ensure that the cap 102 and/or cartridge 126 are firmly pressed against the user's skin. In these examples, the cap 102 may include an additional auxiliary trigger (not shown) which only allows the user to fire the injector device 100 when the sensors detect that the cap is firmly pressed against the skin. For instance, the additional trigger auxiliary trigger will only allow the user to fire the injector device 100 when the pressure sensors detect a minimum threshold amount of pressure applied to the outer rim of the cap 102.

In examples, the cap 102 can be integrated into the injector body 104 when retracted. In one example, the cartridge 126 comprises a retractable cap 102, and the retractable cap 102 covers the trigger button 108 in a retracted position. In some examples, the cap 102 is a “bayonet-style” connector 130, as shown in FIG. 4B.

The vertical alignment feature in the cap 102 may be used in conjunction with the safety switch 128 located in the injector body 104. The safety switch 128 must be activated by means of physical interaction or alternatively by compliance with the vertical alignment feature to allow the user to activate the trigger button 108. Failure to comply with sufficient vertical alignment would prevent the user from accidental firing of the injector device 100, improving the safety of the injector device 100.

The injector device 100 may further include injection indicator 110 symbols, according to example embodiments. As noted above, the injection indicator 110 can be a mechanical screen that provides different states of the injector device 100. For instance, in some examples, the injection indicator 110 can show a gray line or bar to indicate that the injector device 100 is not loaded and has not fired (i.e., ejected the medicine from the vial). The injection indicator 110 can show a red bar on the injection indicator 110 to indicate that the injector device 100 is loaded, but has not fired. And the injection indicator 110 can show a green check-mark to indicate that the injector device 100 has successfully fired. In examples, the injection indicator 110 can reset to the gray bar when plugged back into the base station 106.

### II. Methods of Use

In accordance with the example devices and systems described above, in practice, to use the injector device, the user can first place the injector in the base station.

The user can then place the needle-free cartridge onto the injector.

The user can then use the vial adaptor to connect the cartridge and the vial with the vial facing down for drawing medicine.

Next, the user can select the intended dose on the base station. The station starts the motor to load the spring and overfill the intended dose.

The base station then expels air and excess medicine out of the cartridge.

The user can then remove the vial adaptor and then the injector device.

The user can then place the injector device onto the sanitized injection area.

The user can then press the trigger button to initiate the injection.

The injector device then applies high pressure to medicine within the cartridge and ejects it through the small orifice. The medicine is ejected at a high enough velocity to penetrate the skin of the user and deliver the medicine to the intended area without the use of a needle.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Since many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

What is claimed is:

1. An injection system comprising:
an injector device comprising:
a cartridge;
an injector body coupled to the cartridge at a first end of the injector body, the injector body comprising a plug at a second end of the injector body; and
a base station configured to couple to the plug, wherein the base station comprises a motor, and wherein rotation of the motor draws medicine from a vial into the cartridge, and wherein a volume of the medicine drawn from the vial into the cartridge is in excess of an set dosage.

2. The injection system of claim 1, wherein the base station includes a dosage indicator and wherein the base station is configured to draw a dose in excess of the indicated on the dosage indicator from a vial into the cartridge.

3. The injection system of claim 3, wherein the base station comprises a vibrating motor.

4. The injection system of claim 4, wherein the vibrating motor expels excess medicine and air from the cartridge.
